(19) United States
(12) Patent Application Publication
(10) Pub. No.: US 2009/0117598 A1
(43) Pub. Date: May 7, 2009

(54) Method for identifying compounds that affect a transport of a protein through a membrane trafficking pathway

(76) Inventors: Rainer Fischer, Monschau (DE); Neil Emans, Thimister-Clermont (BE); Stefano Di Fiore, Neuss (DE); Carlo Jochems, Koln (DE); Kurt Herrenknecht, Hamburg (DE); Stephan Hurling, Hamburg (DE)

Correspondence Address: VENABLE LLP, P.O. BOX 34385, WASHINGTON, DC, 20043-9998, US

(21) Appl. No.: 11/665,184

(22) PCT Filed: Oct. 13, 2005
(86) PCT No.: PCT/EP05/55220

§ 371 Date: Jan. 7, 2009

(30) Foreign Application Priority Data
Oct. 13, 2004 (EP) 04024393.3

(51) Int. Cl. C12Q 1/48; C12Q 1/34; C12Q 1/00

(52) U.S. Cl. 435/15; 435/18; 435/4

(57) ABSTRACT

The present invention relates to a method for identifying compounds that affect a transport of a receptor of interest through a specific membrane trafficking pathway mediated by said receptor within the context of a generic membrane trafficking pathway, which generic pathway is not mediated by said receptor, characterised by the following steps:
d) monitoring the transport of the receptor of interest in a cell in the presence of the compound, wherein the receptor is labelled with a first marker,
e) monitoring the transport of a second marker through the generic membrane trafficking pathway in a cell in the presence of the compound,
f) comparing the results obtained from steps a) and b) and thereby identifying a compound affecting the specific membrane trafficking pathway.

Furthermore it relates to the generation of a compound database using the method according to the invention as well as to the compound database itself.

The technical problem underlying the present invention is to find a novel approach to identify compounds that affect the membrane trafficking of a protein of interest and not the housekeeping vesicle transport machinery that typically takes place at the same time.

This problem is solved by the present invention which discloses in its most general sense a method for identifying compounds that affect a transport of a protein of interest through a specific membrane trafficking pathway, especially a protein-mediated membrane trafficking pathway, in cells by identifying the transport of the protein within the context of a generic membrane trafficking pathway, characterised by the following steps:
a) monitoring the transport of the protein of interest in a cell in the presence of the compound,
b) monitoring the generic membrane trafficking pathway in a cell in the presence of the compound,
c) comparing the results obtained from steps a) and b) and thereby identifying a compound affecting the specific membrane trafficking pathway.

The method according to the present invention is particularly suited to be applied in a scenario in which the generic membrane trafficking pathway and the specific membrane trafficking pathway are partially overlapping. Such an overlap may for instance occur if an enzyme is part of both such pathways.

More specifically, the present invention discloses a method for identifying compounds that affect a transport of a receptor of interest through a specific membrane trafficking pathway mediated by said receptor within the context of a generic membrane trafficking pathway, which generic pathway is not mediated by said receptor, characterised by the following steps:
a) monitoring the transport of the receptor of interest in a cell in the presence of the compound, wherein the receptor is labelled with a first marker,
b) monitoring the transport of a second marker through the generic membrane trafficking pathway in a cell in the presence of the compound,
c) comparing the results obtained from steps a) and b) and thereby identifying a compound affecting the specific membrane trafficking pathway.

The underlying principle of the present invention is the identification of a compound as specifically affecting the specific membrane trafficking pathway if it does not substantially affect the transport of the second marker, which may in particular be a fluorescently-labelled second marker. A second marker is typically a molecule known to be up-taken by the cell via the generic membrane trafficking pathway, e.g. a pathway known as fluid-phase endocytosis or as pinocytosis.

The transport of the protein of interest and/or the extent of generic membrane trafficking pathway activity can be quantified using the method according to the invention.

It can be preferred, that steps a) and b) are performed simultaneously, preferably utilizing the same cells in steps a) and b). However, steps a) and b) may also be performed sequentially or conversely. One or more cell samples may be analysed at the same time.

It can be preferred, that the protein of interest is over-expressed in the cells. Wild-type cells may also be used in the method according to the invention.

According to the invention, the protein of interest is expressed as a labelled protein. Especially preferred is a fluorescently-labelled protein, for example a GFP-labelled protein. Generally, a luminescent labelling creating a fluorescent or phosphorescent protein is suitable for the method according to the invention. Other methods of labelling the protein of interest include e.g. affinity tagging.

A second marker, preferably a fluorescently labelled marker, is added to the cells and its transport is studied to monitor the generic membrane trafficking pathway. Preferred soluble markers according to the invention are selected from the group comprising fluorescent dextran, proteins or other molecules known to be up-taken/transported by the cell through said generic membrane trafficking pathway.

The fluorescently-labelled protein of interest and the fluorescently-labelled second marker may differ in their excitation and/or emission wavelength. Such an embodiment is particularly advantageous when conducting steps a) and b) at the same time. In this embodiment, a microscope comprising e.g. two detectors or a detector with spectral resolution may be applied to monitor the emission of said protein and said marker.

It may be also be possible to utilize fluorescent labels which differ in their excitation wavelength. These may be excited by different optical sources, such as lasers of a microscope. In this embodiment, the lasers alternately excite the different fluorescent labels in rapid succession, allowing for time-multiplexed detection of the respective emission signals utilizing a common detector.

According to the method of the invention, a compound is identified as specifically affecting the specific membrane trafficking pathway of a protein of interest if it does not substantially affect the transport of the fluorescently-labelled marker.

Optical, biochemical or physiological techniques for steps a) and b) of the method according to the invention can be employed. Preferably, the optical technique is of spectroscopic or microscopic nature, especially confocal microscopy or multi-photon excitation microscopy.

Employing the method according to the invention allows the monitoring or measurement of the delivery of the protein of interest to the endocytotic pathway as well as to the recycling endosome.

As already mentioned, the generic activity of the membrane trafficking pathway can be monitored or measured. Preferably, the membrane trafficking pathway is selected from the group comprising endocytosis, especially fluid-phase endocytosis, exocytosis and neurotransmission.

It can be preferred that the compounds that are identified using the method according to the invention affect the transport of the protein of interest at defined stages of the trafficking pathway. These compounds can be selected from the group comprising kinase inhibitors and phosphatase inhibitors. Especially preferred compounds comprise inhibitors of protein kinases C, A and G.

The protein of interest whose transport through a plasma membrane trafficking pathway is analysed using the method according to the invention can be a receptor, in particular a member of the family of G-protein coupled receptors. The receptor may be a cell surface receptor which may be a determinant of the hormonal responsiveness of a tissue or organ. Especially preferred is the endothelin A receptor.

The method according to the invention can be performed using live cells as well as cells that were chemically fixated.

The method according to the invention can be used to generate a database containing information on:

a) compounds acting only on a specific membrane trafficking pathway
b) compounds acting only on a generic membrane trafficking pathway
c) compounds acting on both pathways
d) compounds acting on neither pathway

A database which is generated as explained above is also claimed. This database may contain compounds that were found to be acting to a minor degree un-specifically on a generic or specific membrane trafficking pathway. Said compounds can subsequently be structurally optimized to create compounds with an improved specificity.

The method according to the invention will be exemplified using a member of the family of G-protein coupled receptors, namely the endothelin A receptor (ETAR).

G-protein coupled receptors (GPCRs) play a key role in one of the most sensitive mechanisms used by cells to sense and respond to changes in their environment. They regulate a very broad range of responses, and this is reflected in their importance as pharmaceutical targets—50% of drugs are estimated to directly or indirectly target GPCRs. After activation by hormone/ligand binding, GPCRs expend their signalling activity at the cell surface and many are selectively sorted and internalized. Internalization is a key step in receptor resensitization but the mechanisms that regulate internalization of GPCRs are not fully understood. Ligand binding triggers the phosphorylation of the GPCR by a GPCR kinase and initiates the interaction with its adaptor, beta-arrestin, and hence its internalization, after which GPCRs interact with many proteins controlling their trafficking through endosomes. In drug screening, compounds modulating the activity of molecules, such as enzymes, in the pathway downstream from the GPCR are searched for. Such molecules might not only be part of the specific membrane trafficking pathway relating to the GPCR, but might also be involved in generic membrane pathways. It is therefore crucial to distinguish between compounds specific and unspecific mode of action. The present invention provides a solution to this important problem of drug discovery.

Two endothelin receptor isoforms—the type A (ETAR) and B (ETBR) receptor—have been extensively characterized. These GPCRs bind endothelins (ETs), a class of peptide hormones with strong vasoactive properties. The two receptor isoforms have different cell type distribution and physiologies. The A receptor is mainly located on vascular smooth muscle cells; on endothelin binding, it promotes long-lived vasoconstriction. The B receptor is mainly located on endothelial cells; on endothelin binding, it produces a short-lived vasodilatatory response. The B receptor shows very high affinity for ETs and very long binding half-life and, because of this, is involved in clearance of ETs from the blood stream.

The two receptors have different endocytotic trafficking pathways and target destinations. The ETAR is localized on the plasma membrane until stimulated by endothelins, such as endothelin-1 (ET-1) towards which the receptor shows the highest binding affinity. ET-1 triggers ETAR internalization into a peri-centriolar recycling endosome. In contrast, ETBR binds several ET isoforms, such as ET-1, -2 and -3, is constitutively internalized and is not recycled but transported to the lysosome for degradation.

The method according to the invention will be explained in detail in the following examples.

### EXAMPLES

### Materials and Methods

### Chemicals and Reagents

All fine chemicals were purchased from Sigma-Aldrich. Fluorophores and their reactive forms were purchased from Molecular Probes (Eugene, USA) and the nuclear stain DRAQ5 was from BioStatus (Shepshed, UK). 40 kDa BODIPY-Fl-dextran was synthesized from amino dextran using standard protocols. The lyophilized product had a typical labelling ratio of 3 fluorophores·mol⁻¹. Kinase and phosphatase inhibitors were purchased as 95-99% pure 10 mM stock solutions in dimethylsulfoxide or water (Biomol Hamburg, Germany). Stock solutions and formatted assay plates were stored at −20° C.

### Cell Lines and Cell Culture

Hela cells were obtained from the German Collection of Microorganisms and Cell Cultures (Braunschweig, Germany). Hela cells were cultivated in phenol red free Dulbecco's modified eagles medium (Invitrogen; Carlsbad, USA) supplemented with 100% foetal calf serum (Biochrom; Berlin, Germany) and 1% penicillin streptomycin (Invitrogen; Carlsbad, USA). HEK 293 cells were cultivated in Dulbecco's modified eagle's medium/F12 (Invitrogen; Carlsbad, USA) supplemented with 100% foetal calf serum, 1% penicillin streptomycin and 1% genetecin.

A human endothelin A receptor cDNA clone in pCDNA3.1(−) (Invitrogen Carlsbad, USA) was excised via KpnI/HindIII and fused to the egfp coding sequence of the pEGFP-N1 vector (Clontech, Palo Alto USA) by splice overlap PCR using specific primers. The amplified fragment was digested via KpnI/EcoRV and ligated in the pCDNA3.1(−) vector digested with the same combination of restriction enzymes. The resulting pETAR-EGFP DNA was cloned, checked by sequencing and used for transfection of HEK 293 cells using standard protocols. A recombinant clone was obtained through several cycles of genetecin selection and limiting dilutions. For screening, cells were passed onto coverslip bottomed 96 well plates (Greiner; Longwood, USA, or Whatman; Brentford, UK) at a density of 4·10³ cells/well for Hela and 2·10⁴ cells/well for HEK293 cells, 48 hours in advance.

### Cell Imaging

The endothelin receptor translocation assay and the fluid phase endocytosis assay were screened on the Opera ultra-high throughput confocal screening system (Evotec Technologies, Hamburg; Germany). The Opera is a fully automated 3 colour laser excitation confocal system based on an inverted microscope architecture to image cells cultivated in coverslip bottomed microtitre plates. 488, 532 or 633 nm laser excitation is delivered to a dual wheel Nipkow spinning disk that ensures confocality. Images were acquired using an Olympus 20×0.7 numerical aperture water immersion objective mounted on a piezo element for focusing and fitted at the top with a sealing ring and a perfusion system to permit automated water immersion and water withdrawal at the face of the objective. Emitted light from the sample was delivered to a 630 nm dichroic mirror that split the emitted light between two 12 bit high quantum efficiency 1.3 megapixel CCD cameras. All operating parameters for the instrument were controlled by the Opera software running on a 2 Ghz PC connected to a 1 Gbps local network of three image analysis computers. Image alignment, flat field and background correction were automatically performed based on reference and orientation images. Automated experimental acquisition protocols were defined for each screening based on trial samples. 12 bit grey scale image pairs were captured, quantitatively analysed and stored in a compressed format. Images were archived within a 240 GB storage database or automatically exported as 16 bit TIFF files for analysis in Metamorph (Universal Imaging; West Chester, Pa.) or for presentation.

Coverslip grown cells were imaged using a Leica TCS SP confocal using 488 nm and 568 nm excitation and 510-530 and 590-620 nm emission filter settings, respectively. Images were processed and overlaid using Metamorph.

### Cell Based Assay Screening Protocols

Compounds in DMSO or H₂O were aliquoted into 96 well v-bottomed screening plates (Matrix Technologies; Hudson, USA) before screening and stored at −20° C. in the assay format. Compounds were diluted into 1% serum (HEK screens) or serum free (Hela screens) buffered media at the working concentration (0.1 nM to 10 μM) just prior to screening.

For the GPCR internalization assay, recombinant HEK cells grown were plated in 96 well plates and incubated overnight in DMEM/F12 containing 10% FBS. The plates were washed and the cells incubated for 120 min under tissue culture conditions with the compounds in 1% serum medium. The compound solutions were then exchanged for a solution with the same compound concentration and supplemented with 40 nM endothelin-1 and 10 μM Syto60. Cells were incubated for 120 min at 22° C. Plates were imaged by the Opera using 488/633 nm excitation, 3 μm focus height above the coverslip, 250 ms or 500 ms exposure and 510 nm (50 nm bandpass) or 680 (50 nm bandpass) filters, respectively. Typically, 5 image pairs (at 488 and 633 nm excitation) per well were acquired.

For the fluid phase endocytosis assay, Hela cells in 96 well plates were washed and incubated for 120 min with the compounds in serum free buffered medium under tissue culture conditions. The medium was then replaced with serum-free buffered medium supplemented with 1 mg/ml BODIPY-FL-Dextran and 10 μM Syto60. Cells were incubated for 20 min at 37° C., then placed on a cooled block and washed extensively with ice cold phosphate buffered saline 10% w/v bovine serum albumin (Serva; Heidelberg, Germany). Plates were imaged by the Opera using 488/633 nm excitation, 3 μm focus height above the coverslip, 1000 ms or 500 ms exposure and 510 nm (50 nm bandpass) or 680 (50 nm bandpass) filters, respectively.

Cell Based Assay Evaluation

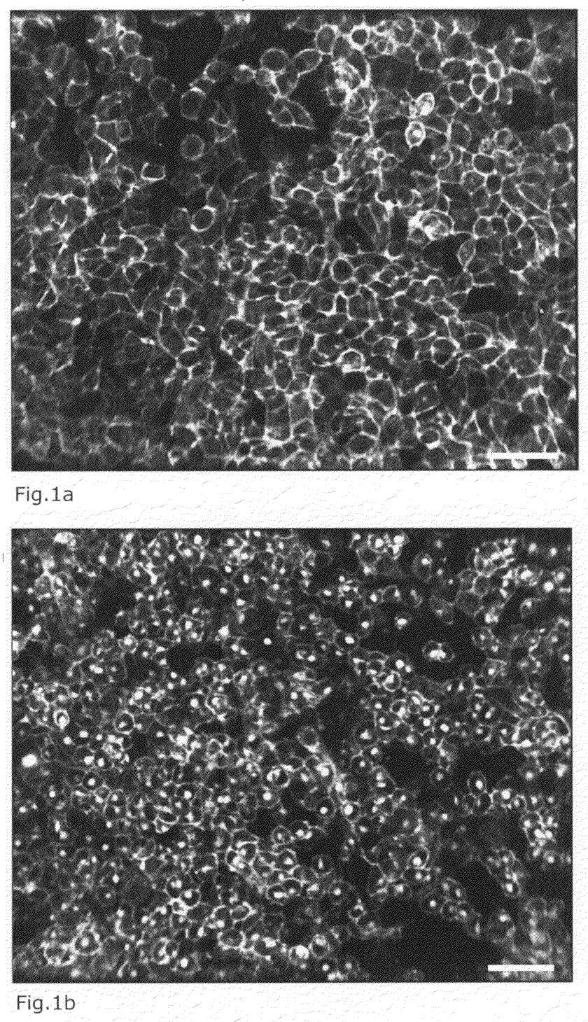

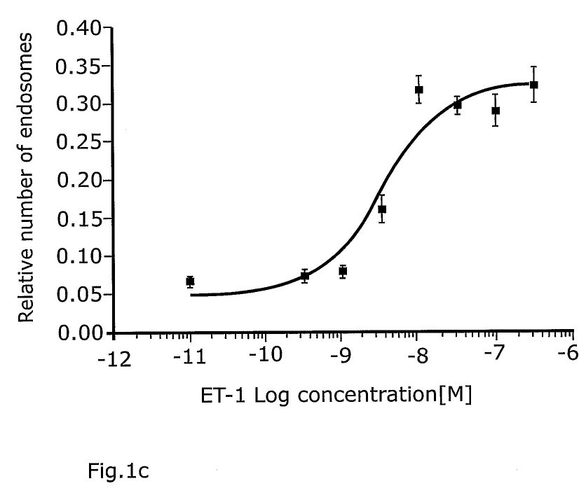

GPCR translocation was evaluated using scripts within the Acapella script player software environment of the Opera. The script measured translocation of the ETAR-EGFP fusion protein from the plasma membrane into recycling endosomes. Cells were first located in the 633 nm excitation SYTO60 image. Red fluorescent nuclei were identified by thresholding and edge seeking and then segmented to create a mask. The number of cells was counted and the nuclei mask used to generate a new cytoplasmic mask. Subsequently, each cell was individually segmented and cells with at least one bright recycling endosome were identified. Image analysis was performed on- and offline and analyses were rejected if the cell count was beneath 100 per field of view. The script passed the acceptance criteria of measuring the EC₅₀ of endothelin stimulation as 3.4 nM, with a Z′ of 0.7 (FIG. 1c).

Fluid phase endocytosis was measured using custom written scripts in Metamorph. Cells were identified in the 633 nm excitation SYTO60 image, the nuclei segmented and cells counted. A mask was generated from this image and used to identify cells in overlay of the 488 nm BODIPY-FL dextran image, to create regions of interest around the cell borders and excise them from the image. Background was deducted from the processed BODIPY-FL dextran image that was thresholded to identify endosomes as individual regions of interest. Image pairs with less than 300 cells per field of view were rejected. This assay had a Z′ of 0.64.

### Example 1

### Measurement of GPCR Internalization and Fluid Phase Endocytosis

The endothelin A GPCR internalization screen was carried out using a stably transfected HEK 293 cell line expressing a fusion protein between the ETAR and a c-terminal copy of the enhanced green fluorescent protein. The ETAR fusion protein expressed at detectable levels in HEK293 cells where it localized to the plasma membrane with hardly any internal labelling (FIG. 1a). After serum deprivation for 16 hours, stimulation of stably expressing HEK293 cells with 40 nM endothelin-1 lead to internalization of the fluorescent ETAR into an intracellular peri-centriolar compartment (FIG. 1b). Co-localization experiments after red fluorescent transferrin internalization confirmed that the ETAR localized to a recycling endosome (data not shown). This internalization event was used as the basis for measuring trafficking of the receptor by image analysis. Serum deprived cells were stimulated with endothelin-1 at several concentrations and simultaneously counterstained with DRAQ5 or SYTO60, which are long wavelength excitable DNA and nucleic acid stains, respectively. The cells were stimulated for 120 min at 22-23° C., then imaged on the Opera automated confocal imaging reader. Images of the GFP and red counterstain fluorescence were automatically recorded and used to determine the number of cells where internalization of the receptor had occurred. The relative number of cells showing endosomal internalization at a range of endothelin-1 concentrations is shown in FIG. 1c; from these data, the EC₅₀ for endothelin was determined to be 3.4 nM. This EC₅₀ value for endothelin-1 activation of the ETAR-EGFP is in agreement with previously reported imaging and non-imaging based measurements for ETAR activation, and validates the assay as a measure of GPCR activation and internalization.

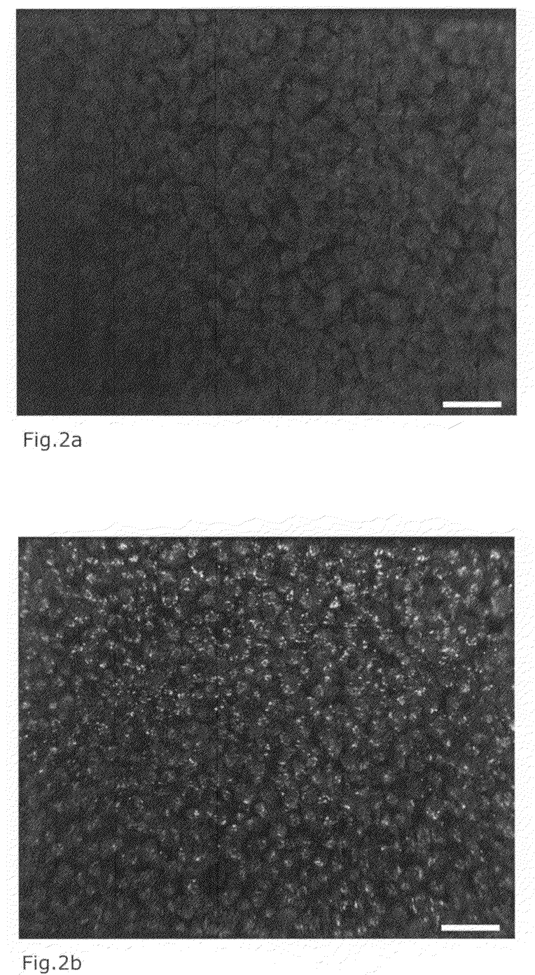

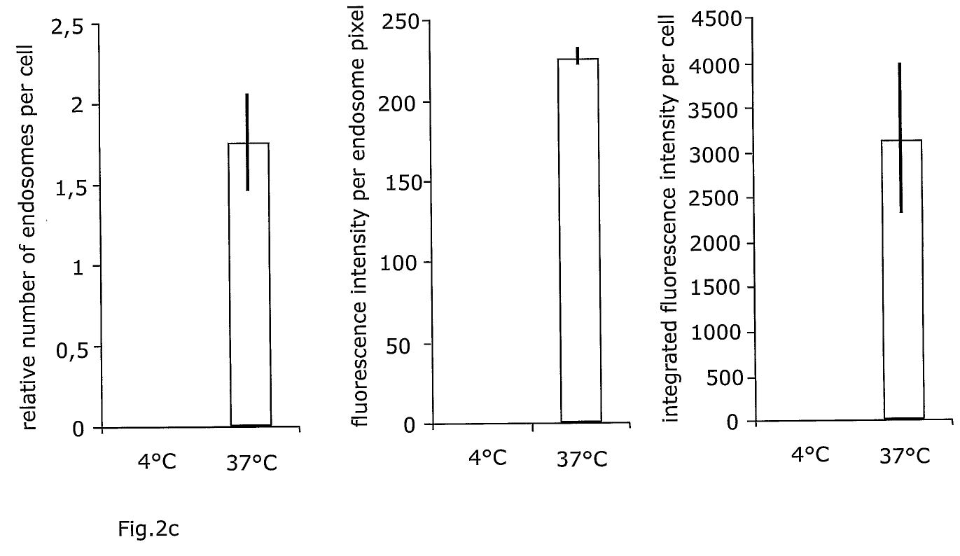

A fluid phase endocytosis assay measured the internalization of green fluorescent 40 kDa BODIPY-FI dextran. Dextrans are ideal endocytotic markers for labelling endosomes and macropinosomes. In this assay, endosomes labelled with fluorescent dextran by internalization were readily identified with the Opera instrument (FIG. 2b). Dextran internalization satisfied all the classical prerequisites for an internalization assay—it was temperature dependent, arrested at 4° C. (FIG. 2a), and required a source of energy in the medium. Endocytosis was quantified by analysis of paired images of cells labelled with green dextran and the SYTO60 nuclear counter stain. The analysis was validated on cells that had internalized green fluorescent dextran for 20 min at 37° C. or at 4° C. (FIG. 2c). This simple one step screen of endocytosis may prove useful for genome wide screening for proteins involved in the pathway using small interfering RNAs.

### Example 2

### Measuring GPCR Internalization

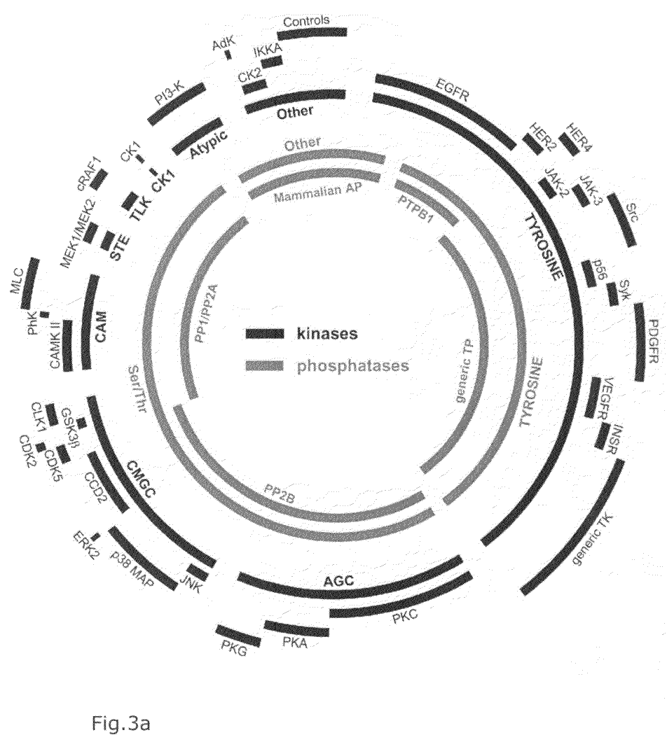

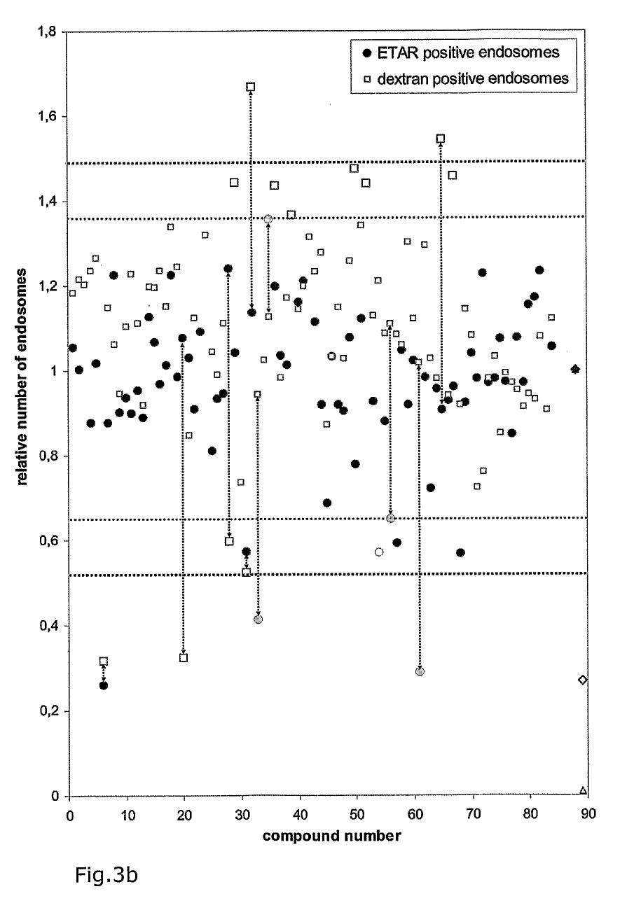

The effect of 84 kinase and phosphatase inhibitors on GPCR endocytosis was screened using the ETAR internalization assay. As shown in the rotary map in FIG. 3a, the collection of compounds included inhibitors directed against kinases of eight out of nine groups/families described in the most recent classification of the human kinome and against three classes of phosphatases.

Cells were pre-treated with 10 μM compound before stimulation with 40 nM endothelin-1 to ensure a uniform and robust internalization response for the receptor. No changes in receptor distribution were detected after compound treatment alone (data not shown). Receptor internalization was measured in cells treated with the compounds by automated image analysis of 5 image pairs per compound per experiment. No internalization was observed in unstimulated or untreated cells, or in cells treated with DMSO alone (FIG. 3b). Internalization of the receptor in stimulated cells was robust, and control-stimulated and control-unstimulated measurements corresponded to the expected values from the titration curve (FIG. 1c).

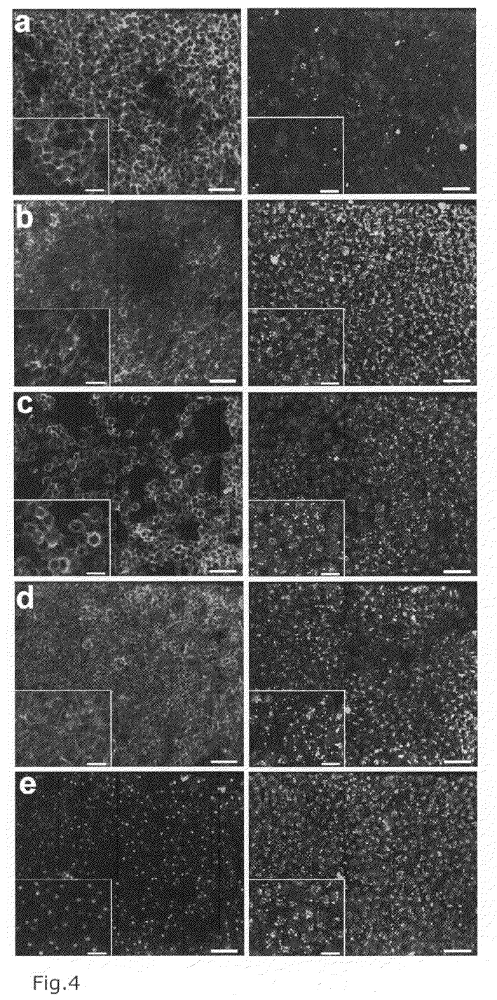

The primary screen used image analysis to identify wells where the relative amount of receptor internalization differed from the control (FIG. 3b). The output of the automated image analysis indicated that some compounds affected GPCR internalization and some had lethal effects on the cells. Primary hits were identified as those compounds that gave a response within or beyond a significance threshold set at two-to-three times the standard deviation of the untreated stimulated control cell population (FIG. 3b). Images of the primary hit compounds were then visually screened to identify changes in receptor distribution and/or trafficking as compared to control cells. The visual screen confirmed the result of the automated analysis and further distinguished hits with actual effects on receptor trafficking from those with partial or lethal effects on the cells. A total of six compounds were selected that altered receptor trafficking. The morphologies observed with 10 μM compound comprised: arrest of the receptor at the plasma membrane (e.g. staurosporine, Ro 31-8220, FIG. 4a, and b, respectively), arrest of the receptor in small vesicular bodies, presumably early endosomal compartments (erbstatin analogue; FIG. 4c) or enhanced accumulation in the recycling endosome (H-89; FIG. 4e).

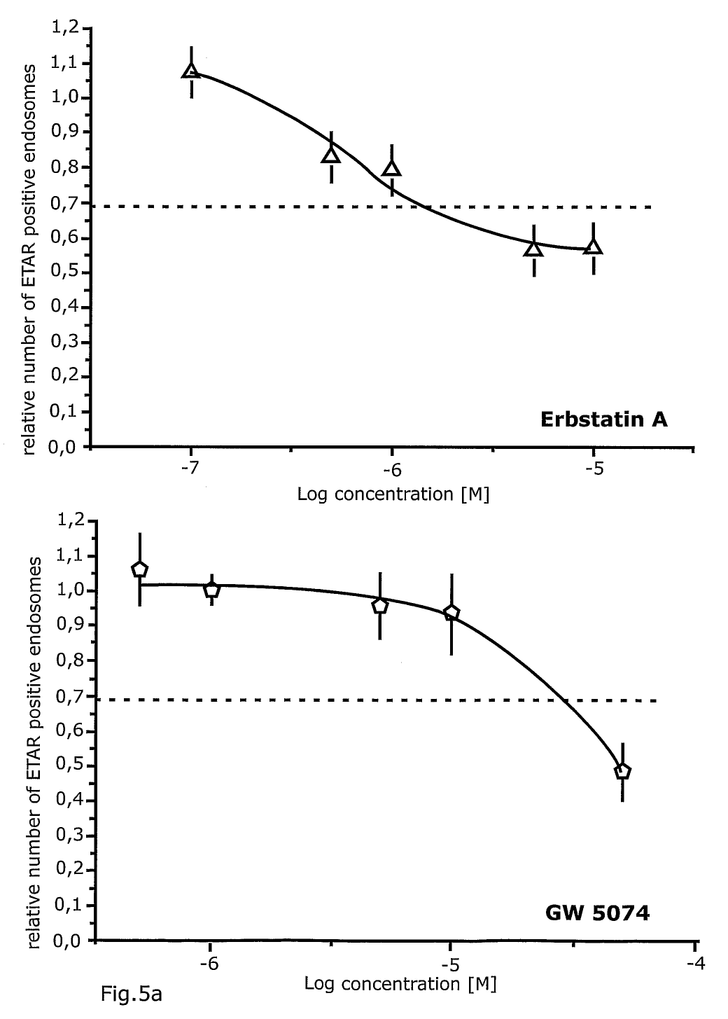

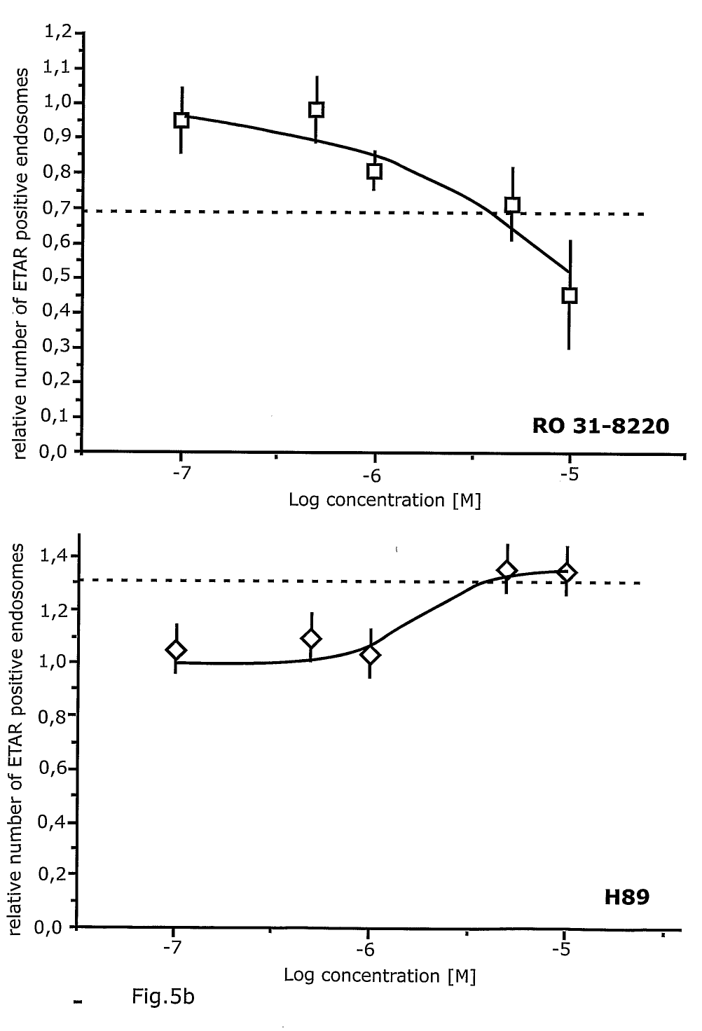

Secondary screening was performed on a subset of compounds including the hits of the primary screen (FIG. 3b). The secondary screen was performed across a range from 0.1 μM to 50 μM to assign dose dependency to the observed morphologies (FIG. 5).

The most effective blocker of GPCR internalization was staurosporine, which had an effective concentration at 100 nM; in contrast GW 5074—a partial hit in the primary screen—had effect at 50 μM (FIG. 5). GW 5074 lead to delayed accumulation of the receptor in the recycling endosomes as judged by altered shape and consistently lower fluorescence intensity of these compartments in comparison to those of untreated and endothelin-1 stimulated control cells (FIG. 4d).

### Example 3

### Screening the Fluid Phase Endocytosis Assay

To identify compounds affecting GPCR internalization and not affecting housekeeping endocytosis, the kinase and phosphatase inhibitor collection was screened against the fluid phase endocytosis assay (FIG. 3b). The effect of the compounds was evaluated. Four compounds (staurosporine, tyrphostin 9, AG-879, GF 109203×) were identified that reduced the number of endosomes (FIG. 3b) and the integrated intensity per cell relative to the control (data not shown). Of these, only staurosporine slightly increased the endosomal pixel intensity. Conversely, some compounds increased the relative number of endosomes of which two (hypericin and genistein) resulted in significantly increased integrated fluorescence intensity per cell. Visual analysis of the cells treated with the hit compounds did not identify any anomalies compared to control cells, except the absence of internalized green fluorescent dextran.

Overlap of the ETAR internalization and the fluid phase endocytosis screen was used to generate differential information from the two assays. This clearly identified molecules whose effect was congruent in both assays (e.g. staurosporine and GF 109203X; FIG. 3b) or those where there was a large difference between the responses of the two assays (e.g. Ro 31-8220, erbstatin A, H-89, GW 5074; FIG. 3b).

1: A method for identifying a compound that affects a transport of a receptor through a specific membrane trafficking pathway mediated by said receptor within the context of a generic membrane trafficking pathway, which generic pathway is not mediated by said receptor, characterised by the following steps:
a) monitoring the transport of the receptor in a cell in the presence of the compound, wherein the receptor is labelled with a first marker,
b) monitoring the transport of a second marker through the generic membrane trafficking pathway in a cell in the presence of the compound,
c) comparing the results obtained from steps a) and b) and thereby identifying a compound affecting the specific membrane trafficking pathway.

2: The method according to claim 1, wherein the transport of the receptor and/or the extent of the generic membrane trafficking pathway activity is quantified.

3: The method of claim 1, wherein the generic membrane trafficking pathway is selected from the group comprising endocytosis, exocytosis and neurotransmission.

4: The method according to claim 1, wherein the receptor is a cell surface receptor and the receptor-mediated membrane trafficking pathway is receptor-mediated endocytosis.

5: The method according to claim 3, wherein the generic membrane trafficking pathway is fluid-phase endocytosis.

6: The method according to claim 1, wherein steps a) and b) are performed simultaneously.

7: The method according to claim 1, wherein the receptor is over-expressed in the cell.

8: The method according to claim 1, wherein the receptor is expressed as a labelled protein.

9: The method according to claim 1, wherein a labelled second marker, is added to the cell and its transport is studied as a tool for monitoring the generic membrane trafficking pathway.

10: The method of claim 8, wherein the labelled protein and the second marker differ in their excitation and/or emission wavelength.

11: The method of claim 9, wherein the labelled second marker is selected from the group comprising fluorescent dextran or other molecules known to be uptaken by fluid-phase endocytosis.

12: The method according to claim 1, wherein the compound is identified as specifically affecting the specific membrane trafficking pathway if it does not substantially affect the transport of the second marker.

13: The method according to claim 1, wherein steps a) and b) are performed by optical, biochemical or physiological methods.

14: The method according to claim 29, wherein the microscopic method is confocal microscopy or multi-photon excitation microscopy.

15: The method according to claim 1, wherein the receptor is the endothelin A receptor.

16: The method according to claim 1, wherein delivery of the receptor to an endocytotic pathway is measured.

17: The method according to claim 1, wherein delivery of the receptor to a recycling endosome is measured.

18: The method according to claim 1, wherein the compound affects the transport of the receptor at defined stages of the trafficking pathway.

19: The method according to claim 1, wherein the compound is selected from the group comprising kinase inhibitors and phosphatase inhibitors.

20: The method according to claim 1, wherein the compound comprises inhibitors of protein kinases C, A and G.

21: The method according to claim 1, wherein the cell is a live cell or a cell that is chemically fixated.

22: A method of generating a database containing information on:
a) compounds acting only on a specific membrane trafficking pathway
b) compounds acting only on a generic membrane trafficking pathway
c) compounds acting on both pathways
d) compounds acting on neither pathway
the method comprising identifying the compounds by the method of claim 1.

23: The method of claim 1 further comprising the step of selecting a compound that affects the generic membrane trafficking pathway only minimally as a starting point for its chemical modification with a view to optimizing the compound's specificity for affecting the specific membrane trafficking pathway.

24: A database containing the results obtainable using the method according to claim 1.

25. The method of claim 4 wherein the cell surface receptor is G-protein coupled receptor.

26: The method of claim 1, wherein the same cell is utilized for conducting steps a) and b).

27: The method of claim 8, wherein the labelled protein is a fluorescently-labelled protein.

28: The method of claim 9, wherein the labelled second marker is a fluorescently-labelled second marker.

29: The method of claim 13 wherein steps (a) and (b) are performed by spectroscopic or microscopic methods.
